(19) United States
(12) Patent Application Publication
(10) Pub. No.: US 2020/0155732 A1
(43) Pub. Date: May 21, 2020

(54) Radiopaque Vascular Prosthesis

(71) Applicant: MicroVention, Inc., Aliso Viejo, CA (US)

(72) Inventors: Hussain Rangwala, Villa Park, CA (US); Ponaka Pung, Signal Hill, CA (US); Shameem Golshan, Laguna Niguel, CA (US); Oanh Nguyen, Tustin, CA (US)

(73) Assignee: MicroVention, Inc., Aliso Viejo, CA (US)

(21) Appl. No.: 16/685,995

(22) Filed: Nov. 15, 2019

Related U.S. Application Data
(60) Provisional application No. 62/768,803, filed on Nov. 16, 2018.

(51) Int. Cl. A61L 31/18; A61F 2/06; A61F 2/966; A61L 31/08

(52) U.S. Cl. CPC A61L 31/18; A61F 2/06; A61F 2/07; A61L 31/088; A61F 2/966

(57) ABSTRACT

A vascular prosthesis utilizing drawn-filled tubing (DFT) wire to aid in visualizing the prosthesis without needing supplemental radiopaque material is described.

### RELATED APPLICATIONS

This application is the nonprovisional of and claims priority to U.S. Provisional Application Ser. No. 62/768,803 filed Nov. 16, 2018 entitled Stent, which is hereby incorporated herein by reference in its entirety.

### BACKGROUND OF THE INVENTION

Vascular prostheses such as stents and stent-grafts are used for a variety of reasons in the vasculature. A non-exhaustive list includes propping open diseased or occluded vessels to promote blood flow, flow diversion involving diverting flow away from target areas such as aneurysms, and retaining material (e.g., embolic material) within a treatment site to promote localized occlusion within a region.

Visualization is important for vascular prostheses so a surgeon can confirm proper placement of the device in the vasculature. Traditional metallic stents utilize a good shape memory material, such as nitinol, so that the stent readily adopts its expanded state upon delivery to the treatment site. The metallic stent is heat-set into an expanded shape such that the stent easily adopts this expanded shape once released from its compressed state in a delivery catheter. However, these metals are not radiopaque which makes imaging a challenge. Though one potential solution is to utilize a radiopaque metal wire in making these stents, in practice these materials are difficult to work with and can often become brittle once heat treatment is applied.

To address this issue, many stents and stent-grafts may utilize one or more radiopaque components to promote visualization so the physician can confirm proper placement of the device in the vasculature. These radiopaque components are distinct from the actual prosthesis itself (e.g. a separate radiopaque layer or component separately attached to the stent). The use of these separate radiopaque components can create complications as they can make the stents thicker, making deployment difficult and affecting if these stents can effectively treat smaller vessels such as those in the neurovasculature. These separate radiopaque components also affect the overall mechanical properties of the stent, creating an engineering challenge.

Drawn-filled tubes (DFT) utilize dissimilar materials including an inner core and an outer jacket. DFT's can be configured with a radiopaque material (e.g. a radiopaque inner core with a non-radiopaque external jacket, or vice versa) to combine the benefits of good shape memory metallic wires along with the radiopacity/imaging benefits of the radiopaque material. These DFT wires can then be used to design a vascular prosthesis which does not require a separate radiopaque material. In this way, the DFT can be used in the metallic layer comprising the stent/stent-graft, enabling visualization of the device without necessitating the inclusion of a separate radiopaque material. However, DFT wires have different mechanical properties compared to traditional metallic wires, creating a different sort of engineering challenge in creating a usable vascular prosthesis which incorporates DFT wires.

There is a need for a usable DFT stent which combines the imaging benefits of DFT while addressing the unique design challenges which DFT wires present.

### SUMMARY OF THE INVENTION

The invention relates to a vascular prosthesis composed of one or more DFT wires, offering benefits in device imaging.

In one embodiment, a vascular prosthesis is composed of a single DFT wire braided back and forth into itself to create a generally tubular shape. In other embodiments, a plurality of DFT wires can be used. In one embodiment, the DFT wire(s) utilize a platinum or tantalum core surrounded by a nitinol jacket.

In one embodiment, a DFT prosthesis utilizes a plurality of end loops on either end of the prosthesis, utilizing shorter loops and longer loops. The shorter loops are sized and angled to enable snug contact with the blood vessel to resist movement at the implant location. In one embodiment, the longer loops utilize a coil or marker coil element to help a delivery pusher grip the prosthesis during prosthesis delivery. In one embodiment, the shorter loops are configured such that they overlap the longer loop coils when the DFT prosthesis is in its compressed delivery state.

The DFT prosthesis contains a plurality of pores along the vessel of the prosthesis. The pores are created by crossing points of the one or more wires comprising the DFT prosthesis. In one embodiment, the pores are sized to allow placement of a microcatheter and/or embolic material therethrough in order to occlude a target treatment location (e.g., an aneurysm).

In one embodiment, a delivery system for a DFT prosthesis includes a pusher comprising two enlarged bands and a recessed region in between. The DFT prosthesis includes a plurality of proximal end loops with longer and shorter loops, where the longer loops utilize coils or marker coils. The coils create a thickened region to engage the enlarged bands to drive the stent during delivery. The shorter loops are sized and positioned such that they overlap a portion of the coils or marker coils when the DFT prosthesis is in its compressed delivery state to prevent the coils from catching when the DFT prosthesis is introduced into, and delivered from, the delivery catheter to thereby enable a smoother delivery. In one embodiment, the pusher system is arranged such that the all of the terminal ends of the shorter and longer proximal end loops are contained in the recessed region between the two enlarged bands.

In one embodiment, a DFT prosthesis utilizes one or more reinforcing elements overlying one or more wire segments of the prosthesis. In one embodiment, the reinforcing element is a wound coil. In another embodiment, the reinforcing element is a tube. The reinforcing element helps increase stiffness along an associated region of the stent and helps the stent to open upon deployment.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 illustrates a DFT wire used in a DFT stent.

FIG. 2 illustrates a DFT stent, according to one embodiment.

FIG. 3 illustrates a mandrel used to wind a DFT stent.

FIG. 4 illustrates a close-up view of an enlarged end region of the mandrel of FIG. 3.

FIG. 5 illustrates loops being wound around the mandrel enlarged end region of FIG. 4.

FIG. 6 illustrates a planar view of a DFT stent end loop configuration, according to one embodiment.

FIG. 7 illustrates a planar view of a DFT stent end loop configuration, according to another embodiment.

FIG. 8 illustrates a planar view of a DFT stent end loop configuration, according to another embodiment.

FIG. 9 illustrates a planar view of a DFT stent end loop configuration, according to another embodiment.

FIG. 10 illustrates a marker coil used on an end loop of a DFT stent, according to one embodiment.

FIG. 11 illustrates a DFT-stent end-loop configuration relative to a pusher system, according to one embodiment.

FIG. 12 illustrates a DFT stent utilizing a reinforcing element, according to one embodiment.

FIGS. 13-14 illustrates a close-up view of the reinforcing element of FIG. 12.

FIG. 15 illustrates a DFT stent weaving pattern, according to one embodiment.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. In that respect, elements and functionality of one embodiment are not necessarily only limited to that embodiment and may be combined with other embodiments shown herein in any manner that would result in a functional embodiment. The terminology used in the detailed description of the embodiments illustrated in the accompanying drawings is not intended to be limiting of the invention. In the drawings, like numbers refer to like elements, including between different embodiments.

The embodiments presented herein deal with creating a usable drawn-filled tubing (DFT) vascular prosthesis (e.g., stent or stent-graft). DFT was discussed above and offers particular advantages in visualization when a drawn-filled tube is created with a radiopaque material. In some examples, these drawn-filled tubes can be used to create wire or filament elements having a radiopaque inner component (e.g., platinum, gold, tantalum, palladium, or other similar materials) and a metallic external jacket/outer component (e.g., nitinol, stainless steel, cobalt-chromium, or other shape memory materials).

With the embodiments of the present invention, the DFT wire is used in a structural layer defining the stent such that the stent is physically composed of one or more DFT wires. In this way, a separate radiopaque component is not needed along the stent since the stent itself is composed of DFT wire and therefore will be capable of easy visualization in-vivo. DFT wire does have its challenges in use since it has different mechanical properties than metallic (e.g., nitinol, stainless steel, cobalt-chromium) wires traditionally used to create stents. Therefore, there are unique design challenges in creating a usable DFT stent. The embodiments of the present invention address these issues by providing unique designs and configurations to create a usable DFT stent.

There are several issues unique to working with DFT wires which need to be considered when designing a DFT stent. One major advantage of using a DFT wire is that the stent itself is visible without the need for a separate radiopaque imaging material. Another advantage is since additional radiopaque components or layers are not needed for visualization, the DFT stents can potentially be sized smaller than conventional stents. This has advantages in enabling easier deployment and in allowing these stents to be fit in (and treat) smaller blood vessels, including those in the distal neurovasculature.

Despite these advantages, there are several design challenges in working with DFT wire and creating a DFT stent. One is that since the DFT wire is a composite of two different materials, it may not have the shape memory qualities of a traditional shape memory metallic (e.g. nitinol) stent, meaning it may not have the same heat-set expansion characteristics as a traditional metallic stent. Second, the inclusion of a radiopaque element or layer on a traditional metallic stent generally increases the stiffness of the stent, which augments characteristics such as apposition and resistance to movement upon implantation. Since a DFT stent would not need this additional radiopaque material for visibility, the resulting reduced stiffness can impact apposition and migration.

Additionally, one surprising issue when working with DFT wires is that these materials often tend to become softer than even a purely metallic shape memory wire once heat treatment/heat-setting occurs. This is generally unexpected since the radiopaque core or inner material (depending on which particular material is used) can be stiffer in comparison to the metallic shape memory outer jacket. However, the inclusion of two separate materials in creating a single wire can alter the material characteristics of the combined wire shape. Due to these characteristics, when DFT wires are used in a stent, mechanical characteristics of the stent have to be adjusted to provide sufficient strength to promote proper deployment of a DFT stent and to promote proper apposition of the DFT stent at the treatment site to prevent stent migration. The embodiments presented herein address these and other issues to create a usable DFT stent.

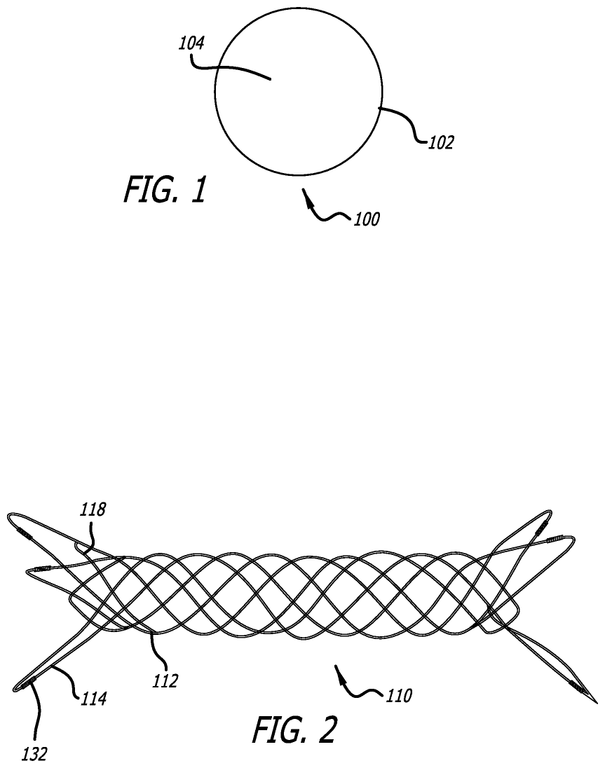

FIG. 1 shows a cross section of a DFT wire 100 utilizing a radiopaque inner wire component 102 and a metallic external jacket 104. The elements that can be used in the radiopaque inner element were discussed above, and include platinum, gold, tantalum, or palladium. The metallic external jacket materials, also discussed above, preferably utilize a strong shape-memory material such as nitinol (generally preferable as a particularly beneficial shape memory material), stainless steel, cobalt-chromium, etc.

The use of the radiopaque inner component allows for visualization while the shape memory jacket allows for good shape memory properties to be imparted into the wire, useful in imparting the stent's heat-shaped expanded configuration. In some embodiments, this configuration can be flipped to utilize a radiopaque outer jacket and a non-radiopaque inner element. In other embodiments, three or more concentrically arranged radial wire elements can be used comprising various combinations of shape-memory metallic and radiopaque components.

The inner component 102 is preferably circular, elliptical, or ovular in shape (e.g., wire shaped) though a variety of shapes can be used, such as rectangular. The external element/jacket 104 can be thought of as a hollow element whose inner diameter is closely matched to the outer diameter of the inner element 102 which surrounds the inner element in a jacket-like configuration. The use of the radiopaque inner element 102 promotes visualization of the DFT wire, while the external jacket 104 (composed of a metallic shape memory material) allows for good pliability of the stent such that the stent can easily adopt a heat-set expanded configuration once deployed or released from a delivery catheter.

During heat-setting, the expanded shape memory is imparted into the material through a heat treatment process. Prior to the heat-setting process or after this, the DFT wire(s) are then electropolished to make them smooth to facilitate implantation in the vasculature.

Various dimensions for the DFT wire can be used depending on the size of the stent, and target treatment procedure (which will influence structural stability needed, stent opening pore size, etc.) In one example, the DFT inner component is made of platinum or tantalum, while the outer component is made of Nitinol-1 or Nitinol-2. In some examples, the overall wire size is about 0.001 inches-0.004 inches, or about 0.0025-0.003 inches. Please note these dimensions are representative of the diameter of the overall wire 100 as shown in FIG. 1. In some examples, the inner radiopaque core is about 0.0005 inches-0.001 inches, or about 0.0008-0.0009 inches.

The overall cross-sectional area of wire 100 is the area of the inner component 104 plus the area of the outer component 102. In one example, the cross-sectional area ratio is such that the radiopaque core is about 10% of the total area of the wire 100. The electropolishing step will further reduce the nitinol jacket area since this is the external section, and in some examples after electropolishing the radiopaque core is about 18-20% of the total area of the wire 100. In some examples, after electropolishing the DFT wire diameter will be about 0.0018-0.0022 inches. In essence, this means that electropolishing will shrink the area of the external jacket and the area of the overall wire cross-section, while the radiopaque inner component area will generally remain the same due to the presence of the overlying shape-memory metal outer jacket.

FIG. 2 illustrates a DFT stent 110, according to one embodiment. The stent 110 is composed of one or more DFT wires 112 wound to create the shape shown, where the DFT wire composition is as discussed above. In one embodiment, the stent 110 is composed of only one DFT wire 112. In another embodiment, a plurality of DFT wires 112 are braided together to create the stent shape. Both ends of the stent utilize flares, including long flares or loops 114 and short flares or loops 118. These flares or loops help provide apposition against the vessel, resist migration at the treatment site, while also helping in the stent delivery process as will be discussed later.

The stent is braided over a mandrel, such that the one or more wires comprising the stent are braided over the mandrel to create the stent shape. In one embodiment, the stent utilizes a single DFT wire 112 where the single DFT wire comprising the stent is braided or woven back and forth over the mandrel to create the shape shown in FIG. 2. This would involve winding in a certain direction (clockwise or counterclockwise) and in a longitudinal manner from one end of the mandrel to the other in an over-under pattern with the other sections of the wound wire, and continuing this process this in a back-and-forth manner from one end of the stent to the other end.

A single wire configuration offers some benefits; for instance, force is transmitted better along a single wire than along a plurality of connected, braided wires since there is no attachment sections which can otherwise affect force transmittal. This is advantageous when pushing the stent through a delivery catheter, and also aids in radially expanding the stent once the stent is free from the overlying delivery catheter. This single wire configuration can also offer advantages when used with a DFT stent, which as discussed above, tends to be softer and less stiff than a typical metallic stent. While this softness is beneficial in terms of the stent conforming to the shape of a tortuous anatomical site, it can also make delivery and apposition more of a challenge, therefore the single wire can transmit and retain force in a more efficient manner.

In another embodiment, the stent utilizes multiple DFT wires 112 (e.g. 2 or more, 4-48, 6-24, 12-24, to provide various examples). The multiples wires comprising the braid are welded or otherwise attached (e.g., crimped, or through a mechanical cap) to each other, such that one wire is attached to another wire, either in one or more locations along the stent, or at the flared end region of the stent.

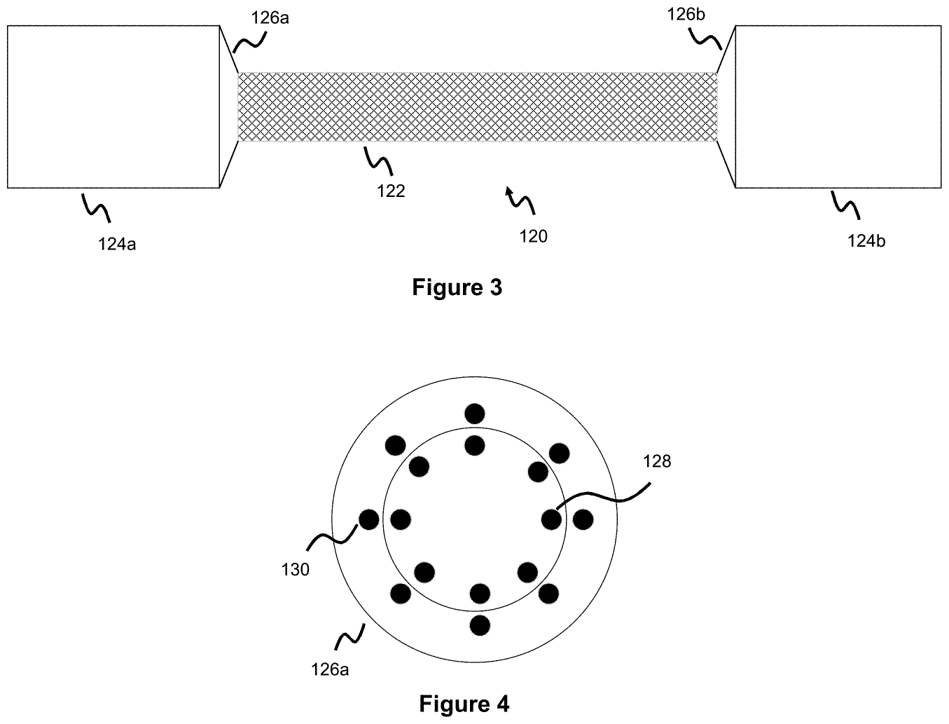

The mandrel 120 used to wind the stent is shown in FIG. 3 and utilizes a cylindrically shaped medial or “middle” section 122 where the majority of the stent is wound, this section creates the cylindrical tubular shape comprising most of the stent. In one embodiment, this medial section 122 can utilize a number of pins around which the one or more wires comprising the stent are wound. In another embodiment, a number of projections, recesses, or laser-cut guide components are utilized to guide the placement of the wire(s) as the wire(s) are wound around the mandrel. In another embodiment, no pins are used, and the user simply tracks the wires in a clockwise, counterclockwise, or mixed manner in a longitudinally circumferential manner around the mandrel.

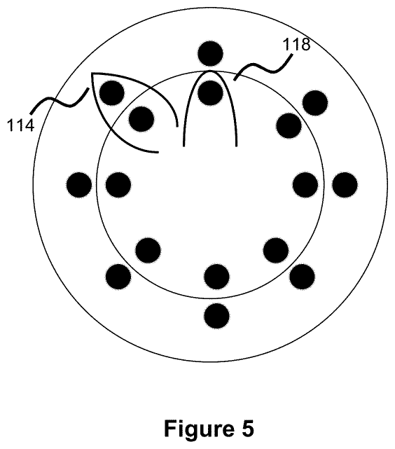

Enlarged mandrel sections 124a and 124b are at either end of this middle section 122. Each enlarged mandrel section 124a, 124b utilizes its own tapered region 126a, 126b which are used to wind the flares or loops 114, 118 of the stent 110. The tapered region 126a (which is similar to the configuration of region 126b) is shown in more detail in FIG. 4. Although this is shown as flat in FIG. 4, a true perspective would show it as tapered such that the radially inner, more-center section projects outward compared to the radially outer, more-peripheral section. The flattened shape as shown is just for ease of illustration.

The tapered regions 126a, 128b include inner pins 128 spread around a radius closer to the center of the tapered region and outer pins 130 spread around a radius farther from the center of the tapered region 126a. The shorter flares 118 of the DFT stent are wound around the inner pins 128, while the longer flares 114 are wound around the outer pins 130. In one example shown, the inner pins are in-line with the outer pins, meaning they share the same line or angle when a line is drawn from the pins to the center of the “flattened” tapered region 126a.

As shown in FIG. 1, each shorter loop is directly next to a longer loop, so each loop is offset from another loop. In practice, this means a short loop is wound on the inner pin 128, wound back through to the other side of the mandrel, comes back and is then wound onto the next outer pin 130 to create a longer loop, is wound over the mandrel, comes back and is wound onto the next inner pin 128 to create a short loop, comes back and is wound onto the next outer pin to create a long loop, etc. With this configuration, a short loop 118 is wound on the inner pin 128, while a long loop 114 is wound on the next outer pin 130, and this pattern continues. This is shown in FIG. 5, where a long loop is next to a short loop. In practice, this means only one pin (either an inner pin 128 or outer pin 130) is used out of each “pair” of pins to wind either a short loop or long loop.

In the context of FIG. 4, eight sets of inner and outer pins are used meaning this pattern could create 4 short loops and 4 long loops, where each loop is offset an equidistant amount (e.g., 45 degrees whereby the short loop is offset by 45 degrees from the adjacent long loops, and vice versa). The pin “pairs” of inner and outer pins are preferably equally spaced, so in the context of FIG. 4 each pin “pair” is spaced by 45 degrees from the adjacent pin “pair”, meaning each loop is spaced by 45 degrees from every other loop. To create a 6-loop configuration of 3 short loops and 3 long loops, 6 “pairs” of pins spaced by 60 degrees from each other would be used on the tapered region 126a, 126b.

The tapered section of the mandrel is used to create the flares, as discussed above. In one example, this taper is about 50 to 60 degrees, or in a more particular example about 60 degrees. In the context of tapered section 126b, this angle would represent the angle between the tapered section 126b and a horizontal line drawn to the right of the tapered section (e.g. a horizontal line drawn through the center of enlarged mandrel 124b). This angle would represent the approximate angle of the plane defined by stent loops 114, 118, or in other words the degree of the bent angle defined by the stent loops. Therefore, the short loops and long loops would be tapered about 50-60 degrees, or in a more particular example about 60 degrees. Different embodiments can utilize different dimensions; however, this angle can have particular utility in allowing the short loops at a particular size profile to contact the vessel wall without collapsing, thereby promoting proper vessel apposition and providing a firm anchor point in the vessel. In other words, this angle is carefully calibrated to maximize apposition force for the stent against the vessel wall, given the unique characteristics of the DFT stent.

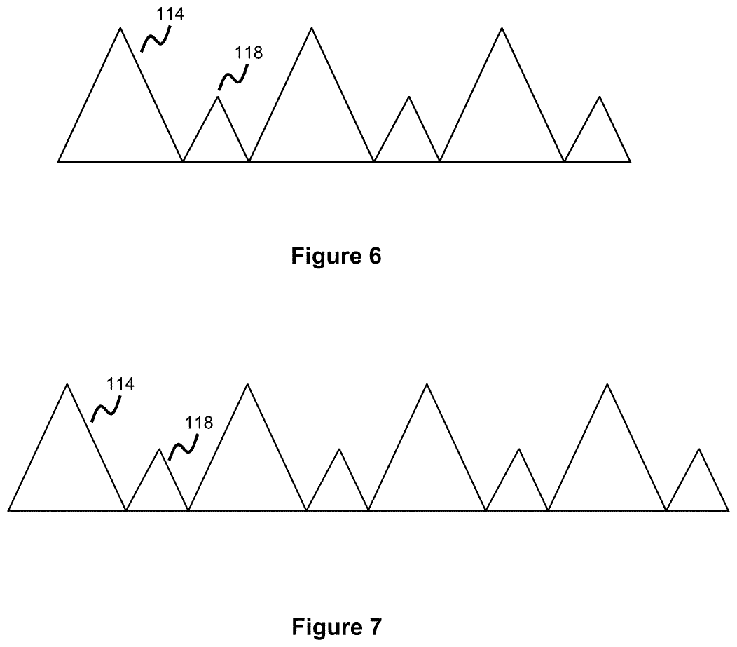

FIGS. 6-9 show the flare or loop configuration at the ends of the stent in more detail. Note, the FIGS. 6-9 show the flares in a linear type of configuration, shown for ease of illustration. These Figures can be thought of as representing the circumferential area formed by the stent ends if one cut was made and then the flares were laid flat along a single plane. Each long loop 114 is next to a short loop 118 such that a long loop 114 will have a short loop 118 on either side of it, while a short loop 118 will have a long loop 114 on either side of it.

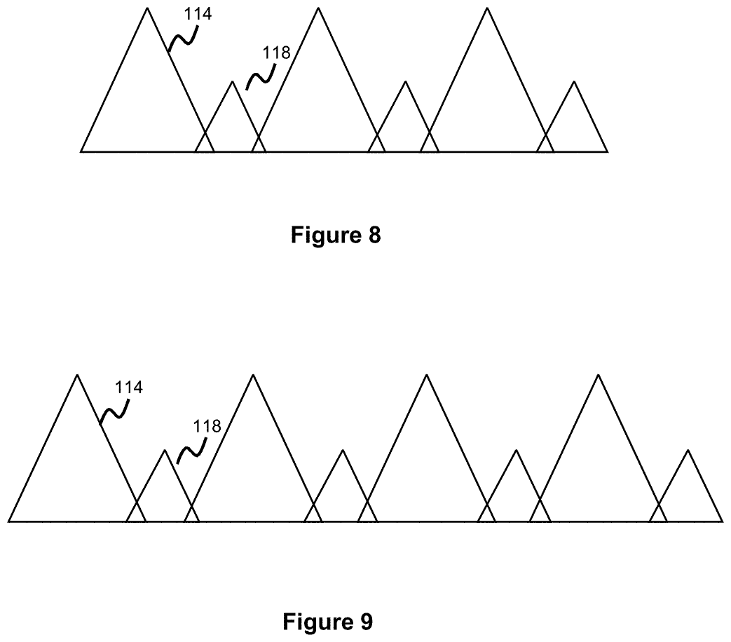

FIGS. 6 and 8 show a 6-loop configuration where each end of the stent has three long loops and three short loops, arranged in an alternating manner. FIGS. 7 and 9 show an 8-loop configuration where each end of the stent has four long loops and four short loops, arranged in an alternating manner.

In FIGS. 5 and 6, the loops/flares are placed substantially next to or aligned with each other in one embodiment of the flare/loop configuration. In FIGS. 7 and 8, the loop/flares overlap a bit, in another embodiment of the flare/loop configuration. This overlap can be created during the heat treatment process, or this overlap can naturally happen due to the wire(s) comprising the loops contacting adjacent wires/loops during stent expansion, resulting in an overlapping configuration. Please note different numbers of flares/loops are possible, for instance each end of the stent can have 4-24 loops.

The short flares 118 are preferably sized to be similar to the diameter of the vessel being treated. In this way the short flares 118, when fully expanded, will directly contact the vessel wall, offering apposition and helping to provide a restraining force to prevent migration of the stent. In this way, the short flares 118 provide support while not collapsing as a result of oversizing relative to the vessel diameter.

Since DFT wires are generally softer than their shape memory metal counterparts, as discussed above, the short flares 118 are carefully calibrated to promote vessel apposition and thereby help prevent movement of the stent once implanted. The short flares 118 have a certain angle (discussed above), length, and shape to maximize apposition force against the vessel wall such that these short flares do not compress downwards (e.g., when oversized to a significant amount compared to the vessel size).

As discussed above, the long flares 114 and short flares 118 can each be oriented at about a 60-degree angle (relative to a horizontal plane extending through the axial/radial middle of the stent). The flare/loop sizes can vary based on the size of the stent as well. In various examples, the stent is sized from about 2.5-5 mm in diameter. This particular size would fit neurovascular arteries, which are smaller than arteries in the majority of the vasculature, and have benefit as a scaffolding stent used to provide support against a neck region of an aneurysm for subsequent devices (e.g. embolic coils, or other occlusive agents) used to fill the aneurysm. Proper apposition of the stent is key in this target therapeutic regimen to ensure the stent does not migrate away from the aneurysm site, which could then allow embolic material to migrate when left without a supporting scaffold.

In some examples, a stent with a fully expanded/deployed width of about 0.1 inches (about 2.5-3 millimeters) has short flares 118 sized about 0.015-0.025 inches and long flares 114 sized about 0.04-0.05 inches; a stent with a fully expanded/deployed width of about 0.12 inches (about 3-3.5 millimeters) has short flares sized about 0.015-0.025 inches and long flares sized about 0.04-0.05 inches; a stent with a fully expanded/deployed width of about 0.14 inches (about 3.5-4 millimeters) has short flares sized about 0.015-0.025 inches and long flares sized about 0.04-0.05 inches; a stent with a fully expanded/deployed width of about 0.162 inches (about 4-4.5 millimeters) has short flares sized about 0.015-0.025 inches and long flares sized about 0.04-0.05.

Please note the fully expanded/deployed width of the stent represents the outer diameter of the tubular portion (i.e. not including the flared end portions) of the stent while the flare lengths represent the length of the flares as they extend from this tubular portion. Further note that the relatively consistent sizing of the short and long flares despite the size of the stent, which in turn are relatively small compared to the overall stent diameter, ensure that the stent can maintain proper contact with the vessel wall to support apposition (via the short flares 118 which are meant to make direct contact with the vessel), while further ensuring the longer flares 114 (which are slightly oversized compared to the blood vessel diameter) do not collapse that much as they are not significantly oversized. This relative sizing also helps to ensure a smoother delivery since the loops do not significantly overhang the stent, resulting in less contact friction against the catheter. The stent lengths can vary based on the sizing and intended use (e.g., size of the treatment area, such as aneurysm, being treated). In some examples the overall stent length can range from about 0.27 inches (about 7 millimeters) to about 0.73 inches (about 18.5 millimeters).

A stent's “working length” refers to the portion of the stent that can be used for its intended treatment purpose. Note, with the ranges given above, the long loops 114 are not drastically oversized relative to the short loops 118 and the short loops 118 are not drastically oversized compared to the rest of the stent. In this manner, in some embodiments, the stent's working length can also include the portion of the stent including the short loops 118, thereby increasing the proportion of the stent that is available to perform a procedure. By way of example, the short loop 118 size range given above would add about 1 mm overall to the “working length” of the stent. For a stent length of 7 mm (representing a lower end of the range given), this 1 mm increase can be significant. Even for a stent length of 18.55 mm (representing an upper end of the range given), this 1 mm is still relatively significant.

The earlier description discussed some of the softness characteristics of the DFT stent. Part of this is due to the lack of a radiopaque component being added to the stent (e.g., either a radiopaque wire wounded through the stent, a separate radiopaque layer, or radiopaque elements being added to selective portions of the stent) and how the inclusion of these layers on traditional stents tends to increase the associated stiffness, as well as observable phenomena involving metallic working and heat treatment of DFT wires.

Due to this increased softness, delivering a DFT stent may require additional force in order to track the DFT stent through the overlying delivery catheter. The preceding description discussed the sizing of the short flares 118 and of the long flares 114, and how they are sized relatively similarly such that the long flares 114 are not significantly longer then the short flares 118. One additional advantage to this design is that the short flares 118 and long flares 114 sit relatively close together when the stent is in the collapsed, deployment state when delivered through the overlying catheter.

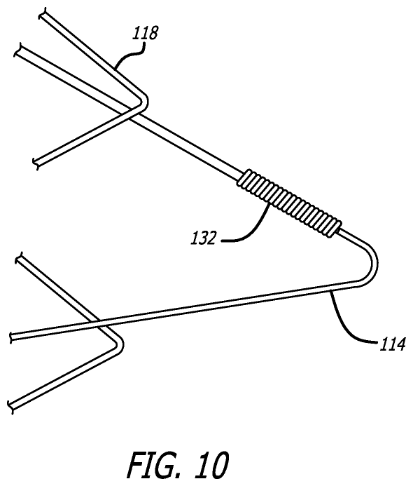

FIG. 10 shows the configuration of a longer flare 114 in more detail. The longer flares 114 include a wound marker coil 132, wrapped around the structural DFT wire comprising the stent. The shorter flares 118 may optionally include a marker coil 132 as well. The marker coil 132 can be a wound wire of tantalum, platinum, palladium or gold wound around the structural DFT wire of the stent. The marker coil(s) 132 on the longer flares 118 are not necessarily being used for visualization purposes since the entire stent is radiopaque (although the enhanced radiopacity may help visualize the two ends of the stent in more detail). Here, they are being used to help grip the stent during delivery, as will be explained.

The use of a radiopaque marker coil 132 on the flares of the stent provides various benefits. Although the stent itself is composed of DFT wire and therefore radiopaque, the inclusion of the marker coil 132 along the flares helps visualize the ends of the stent. In this way, the ends of the stent can stand out and the physician or operator can determine where the ends of the stent are compared to the target region to confirm placement. This is especially useful in situations where a particular facility utilizes relatively poor imaging technology as it at least allows the ends of the stent to be more visible.

The use of a coil 132 as an element has some benefits for DFT wire given its softness, as a wrapped coil typically exerts less force on the underlying wire. Nonetheless, in some embodiments the marker element 132 can take on the form of a tube compressed over the loop section. This might make sense where the DFT wire is particularly thick or the overall diameter of the DFT stent is sized smaller such that there is more strength built into the DFT stent.

In some embodiments, the marker element 132 can be non-radiopaque, for instance where the stent will be oversized compared to the target area and many of the DFT components will overlap. In this situation, having non-radiopaque ends may actually help visualize the ends of the stent better. Therefore, in some embodiments, the marker element 132 can take on the form of a nonradiopaque metallic (e.g., nitinol, stainless steel, or cobalt-chromium) coil or tube.

As will be explained going forward, the marker coil element 132 has particular benefit in increasing the thickness of a loop section to help engage the stent during the delivery process. In this way, the marker coil 132 also functions as a strut thickening element. In these ways, marker coil 132 can also be considered as a non-radiopaque coil, a radiopaque or non-radiopaque tube, and/or a thickening/reinforcing/enlarged member 132 to increase the strut thickness of a portion of the loop section of the DFT stent. In other words, any of these terms can be used to describe element 132, in various embodiments.

FIG. 2 shows the marker coils 132 along the longer flares 114. Note, although not shown, the short loops 118 can also utilize the marker coils 132. In one example one marker coil 132 is used on each long flare 114, in another example two marker coils 132 are used on each flare (one on each part of the “V” comprising the flared shape), in another example one marker coil 132 is used on each short and long flare 114, 118, and in another example two marker coils 132 are used on each short and long flare 114, 118.

The long flare 114 marker coil 132 configuration is shown in more detail in FIG. 10, where FIG. 10 represents the expanded configuration of the stent (similar to FIG. 2). As mentioned before, the opposing segment of the “V” type shape on longer flare 114 can also utilize a marker coil 132 though this is not illustratively shown.

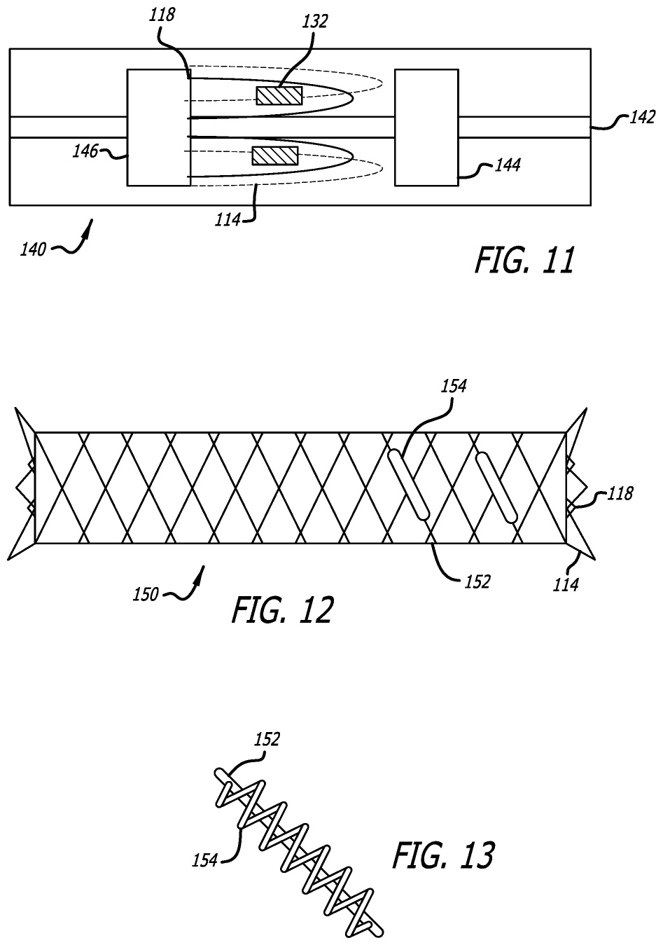

The collapsed configuration of the stent is shown in FIG. 11—in particular the proximal end loops section of the stent, when the stent is restrained within a delivery catheter. Pusher 142 is used to push the stent through a catheter 140. The pusher has a proximal end that the user can push/pull to manipulate the stent, which is connected to the pusher 142, through and out of the catheter 140. The pusher 142 includes a pair of enlarged bands 144, 146 along a distal portion of the pusher 142. In one embodiment, the enlarged bands 144, 146 are radiopaque (e.g. tantalum, gold, platinum, or palladium) to aid in visualization during delivery, and therefore function as marker bands. In some circumstances (based on, for example, sizing of the stent or imaging technology), the inclusion of so much radiopaque material (recall, the entire stent is radiopaque due to the DFT wire) may make visualization more difficult since little will appear discrete. Therefore, in some embodiments the enlarged bands 144, 146 are non-radiopaque (e.g., nitinol or stainless steel).

When the stent is restrained within the delivery catheter 140, the proximal loops of the stent are arranged as shown in FIG. 11, where all of the short loops 118 (shown as the solid loops) and long loops 114 (shown as the dashed loops) at the proximal end of the stent are contained within this region defined between the two enlarged bands 144, 146.

Note, various stent embodiments have various combinations of short and long loops as discussed above, and for ease of illustration 2 short loop and 2 long loops are shown, but this is meant to represent that every proximal loop (both long and short loops) would be constrained within this enlarged pusher band region. In other words, if a stent configuration utilizes 6 proximal loops (3 short, 3 long) all 6 of these proximal loops would sit within this region/space defined between the two enlarged bands 144, 146. Similarly, if a stent configuration utilizes 8 proximal loops (4 short, 4 long) all 8 of these proximal loops would sit within this region, etc.

The marker coils 132 on the long loops serve a key function since they help keep the loops constrained within this region, while also ensuring the enlarged bands 144, 146 can engage the stent. The marker coils 132 on the loops (note, they are shown only along one portion of the long loops, although each loop can utilize two marker coils 132) provide an enlarged contact surface to help engage the stent. In one embodiment (shown in FIG. 11), the proximal band 144 engages the ends of the longer flares 114 to move the stent forward and the distal band 146 engages the marker coils 132 to proximally retract the stent. In the context of FIG. 11, pushing the stent forward will involve moving the stent towards the left, so the proximal band 144 will engages the terminal ends of the long loops 114. Retracting the stent will involve moving the moving the stent towards the right, so the distal band 146 engages the marker coils 132 to engage the stent. Although marker coils 132 are only shown on the longer loops 114, different embodiments can utilize the marker coils 132 only along both the longer loops 114 and the shorter loops 118.

The fact that all of the proximal loops sit within the region between the pusher's enlarged pusher bands 144, 146 provides a number of benefits. For instance, the respective enlarged band will likely contact more than one marker coil since all of the loops/marker coils sit within this region, thereby providing more pushing force as the respective enlarged band engages the marker coils to engage and drive the stent motion. The augmented pushing force has benefits in helping to deliver the stent, especially given the material characteristics of the DFT stent described above. Furthermore, the stacked configuration (whereby all the looped ends sit within this region) also provides a backup system in case one of the stent loops becomes disengaged with respect to the pusher 142, such that the respective enlarged pusher band 144 or 146 can still contact one of the other proximal loops/marker coils to engage the stent.

Please note that since the short loops 118 are shorter than the long loops 114, the short loops 118 will always be slightly offset compared to the long loops—as is shown in FIG. 11. However, in configurations where the short loops 118 also utilize a marker coil, the marker coil position can still be adjusted (for instance, the marker coil can sit, comparatively, closer to the “end” of the short loop) such that the enlarged band 146 can also engage the marker coils 132 of the short loops 118 to engage the stent. In this manner, distal enlarged band 146 can either engage just the marker coils 132 of the short loops 118, just the marker coils 132 of the long loops 114, or the marker coils 132 of both the short loops 118 and the long loop 114.

The stacked configuration where all the loops are contained within the region defined between pusher bands 144, 146 has further advantages. For instance, in many configurations the pusher band will engage the marker coils 132 of the longer loops 114 since these loops 114 are larger than the shorter loops 118 (meaning they have a larger surface area and extend a greater distance). The stacked configuration helps ensure the marker coils 132 along the longer loops 114 are partly protected by the shorter loops 118, thereby reducing the chance that the marker coils 132 snag against the delivery catheter—either during placement from an introducing apparatus into the catheter, or delivery from the catheter into the blood vessel. In the context of FIG. 11, this protection is provided by the short loops 118 actually sitting over or around the marker coil 132 of the long loop, so as to protect it.

Another configuration utilizes an arrangement whereby the ends of the proximal loops are all stacked or constrained within the region defined between the pusher bands 144, 146 but where the proximal band 144 engages the marker coils 132 to drive the stent forward, instead of engaging the ends of the long loops 114. In this configuration, the position of the long loops 114 is different from what is shown in FIG. 11—instead, a portion of the long loops 114 would slightly overhang the enlarged bands 144, 146 whereby the proximal band 144 would engage the marker coil 132 rather than the ends of the long loops 144 to drive the stent. In some embodiments, the short loops 118 can also utilize marker coils 132 and these marker coils can be configured such that the enlarged band 144 makes contact with these marker coils as well.

By way of example, the space or gap between bands 144, 146 (See FIG. 11) is about 1.5-2 mm (about 0.06 to 0.08 inches) in length. Note, since there is not a significant length difference between the long 114 and short 118 loops (sizing dimensions were mentioned earlier, by way of example the short loops are about 0.015-0.025 inches while the long loops are about 0.04-0.05 inches), such a spacing between the enlarged bands will leave sufficient room for all of the terminal ends of the proximal (short and long) loops 114, 118 to sit within this region while the stent is in a delivery configuration.

Please note the configuration shown in FIG. 11 and described previously with regard to how all of the terminal ends of the proximal loops at the proximal end of the stent are contained in the region between the two enlarged bands 144, 146 is described as having particular utility in augmenting delivery force which is beneficial for delivering DFT stents which tend to be softer than conventional stents. Note, this same approach can also be used to deliver non-DFT stents in situations where the augmented delivery force would be beneficial. Therefore, this delivery configuration can be used on either DFT stents or non-DFT stents.

The earlier description discussed how, in many, cases DFT stents may be less stiff than traditional stents. This is due to some of the properties of DFT as well as how the lack of a separate radiopaque material (which tends to be stiff or brittle) can leave the resultant DFT stent softer or less stiff than traditional stents. Softer/less-stiff stents have some advantages in terms of being flexible and able to conform to tortuous regions. However, softer/less-stiff stents may also have less retention strength and can be difficult to fully open or deploy in particular circumstances, e.g., opening across a bend of a tortuous vessel where there are complex forces being applied to the stent. Furthermore, DFT stents may have less built-in shape-memory than traditional stents since the DFT wire-cross section also contains a non-shape memory component, making opening the stent a challenge in some circumstances.

These complications are magnified at a proximal end of a stent which is the last region of the stent to be exposed/expanded upon deployment from the delivery catheter. These complications are also magnified for larger stents, which require more radial force and built-in shape memory to properly expand. The design of the long and short flares, as discussed above with regard to the angles, lengths, and shapes of the flares, is important to help maximize apposition force against the vessel wall. The discussion below discloses ways to augment opening strength in one or more regions of the DFT stent.

FIG. 12 shows one embodiment of a DFT stent 150. As in the previous embodiments the stent utilizes short 118 and long loops 114 at each end of the stent. One or more wires 152 are wound to create the stent shape. One or more regions of the stent 152 include reinforcement elements 154 to introduce increased strength and stiffness along the stent.

In braided stents, it can be difficult to fully expand the proximal end of the stent once the rest of the stent is deployed. This is due to the high forces associated with stent deployment in the vasculature and can be pronounced in more tortuous anatomies. This problem is magnified as stents are designed to be less stiff and more flexible. Therefore, introducing the reinforcement elements 154 along a proximal region of the stent will help augment opening force along this region, promoting ease of deployment.

The reinforcing element 154, in one embodiment, comprises a coil as is shown in greater detail in FIG. 13, where the reinforcing coil is wound around the DFT wire 152 of the stent. In other embodiments, the reinforcing element 154 can comprise a tube that is placed over the DFT wire 152 along one or more regions of the stent. In one embodiment, the reinforcing element 154 is attached to the wire (e.g. via adhesive or welding) to fix the location. In another embodiment, the reinforcing element 154 is not attached and is free to move. In another embodiment, the reinforcing element 154 is another linear wire element which is attached to a portion of the DFT wire 152 to “thicken” the associated DFT wire segment.

The reinforcing element 154, in one example, is made of a strong shape memory material. A preferred example is nitinol (e.g. either a nitinol coil or a nitinol tube), but other examples can include cobalt-chromium or stainless steel.

Where the reinforcing element 154 is a coil, as is shown in FIG. 13, this coil would have an associated stiffness or k-value associated with it. This stiffness/k-value would depend on a number of attributes including the material composition, the thickness of the coil, and how close wound the reinforcing coil is (i.e., the pitch). A higher k-value could be effected, for instance, by utilizing a relatively stiff material (e.g. radiopaque material such as gold, platinum, tungsten, palladium, tantalum, or non-radiopaque metals that are stiff), by using a closely-wound pitch for the coil, and/or by adjusting properties of the coil (e.g., the thickness of the wire comprising the coil, the overall width of the coil, and the overall length of the coiled reinforcing element 154).

The wire portion 152 which is underneath the reinforcing element 154 will have its own associated stiffness of k-value, as the wire forming the reinforcing element 154 will have its own corresponding “springiness” due to being wound in a helical, longitudinal manner along the stent. Note, this “springiness” will increase as the stent is compressed and helps to propel the stent open upon deployment. The k-value of the wire 152 will depend on the associated stiffness of DFT wire, the diameter of the wire, and the pitch of the wire comprising the DFT stent (in other words, the helical/longitudinal wind pattern used to mechanically wind the stent).

The stent region shown in FIG. 13, where the reinforcing coil 154 sits over the wire 152, can be thought of as two parallel springs and Hooke's law would yield a corresponding stiffness. Where the wire 152 has an associated stiffness k₁ and the reinforcing coil 154 has an associated stiffness k₂, the overall stiffness of this region will then be (k₁+k₂), in other words the combined stiffness will be higher. In this way, the reinforcing element 154 serves to increase the associated stiffness at that region. This increased stiffness has certain advantages, for instance strengthening a particular region of the stent to augment deployment force (helping the stent open) and promoting apposition against the vessel wall along the reinforced section.

Another advantage is that the augmented stiffness and the enhanced area that the reinforcing element takes up across the underlying wire will help adjacent cells of the stent open. If adjacent cells cannot sufficiently open, these cells will contact the reinforcing element (which has a higher surface area than the underlying and surrounding wire 152), and this contact force can help these other cells open.

The reinforcing element 154 can be placed in one or more regions along the DFT stent. For instance, it can be placed in roughly equidistant intervals (or alternatively, in random locations) over the length of the stent to promote a consistent expansion and consistent enhanced stiffness across the entirety of the stent. Alternatively, it can be placed along solely the proximal section of the stent (as shown in FIG. 2), in one or more locations along the proximal section in order to enhance strength and opening in the proximal region of the stent.

The reinforcing element 154 can be added in a variety of ways to the DFT wire of the stent. The following techniques can be used regardless of whether the DFT stent comprises solely one DFT wire, or a plurality of DFT wires. In one embodiment, the reinforcing element 154 is slid over the respective wire segment before or during the winding procedure used to wind the stent.

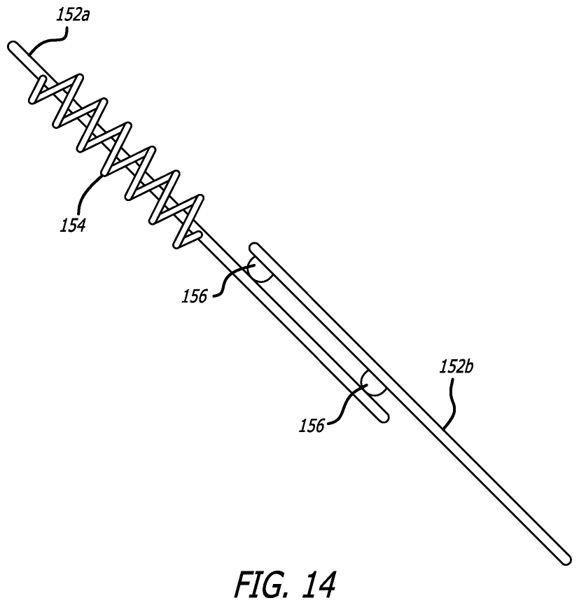

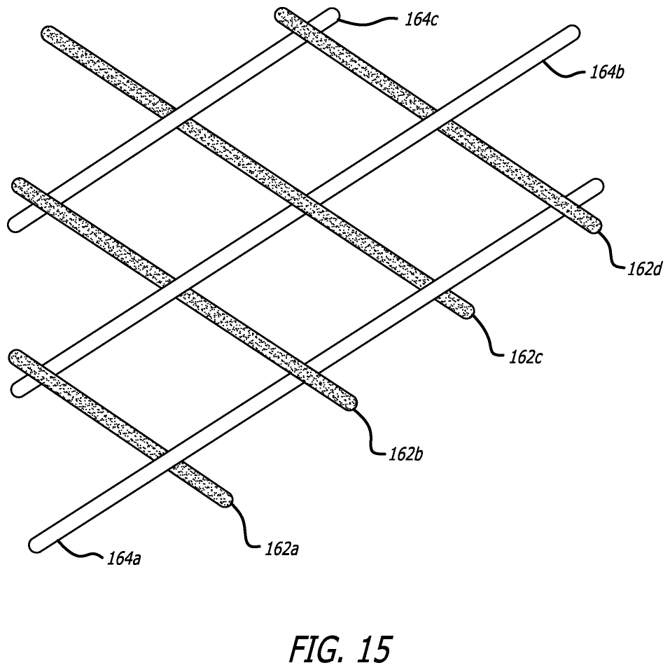

In another embodiment, the wire can be cut near the region where the reinforcing element 154 is added to the wire, and once the reinforcing element 154 is appropriately placed, the wire is then soldered or welded to the other cut section of the wire to reattach the two wire segments. This is shown in FIG. 14, where wire 152 comprises two segments 152a, 152b connected at locations 156. The two wire segments 152a, 152b can either represent a location where one wire is cut into two segments which are then reattached or can represent the location where two separate wires are attached/connected near the reinforcing element 154. One advantage of placing this wire attachment location near the reinforcing element 154 is that this will thicken the associated wire segment, which can help keep the reinforcing element 154 in a particular location and keep it from moving around.

In some examples, the reinforcing element 154 or an increased number of reinforcing elements 154 can be used on larger DFT stents (e.g., those about sized 4-4.5 mm and above) since these stents may be harder to fully open. In various configurations, the reinforcing element can be added to the proximal ⅓ of the stent by loading the reinforcing element 154 over two (as shown in FIG. 12) or more winding locations. In one example, the reinforcing element 154 is a nitinol coil having an inner diameter of about 0.003 inches and an outer diameter of about 0.0065 inches. Where plural reinforcing elements 154 are used, they can be spaced in various ways, for instance one wire wind can separate two elements 154 (shown in FIG. 12), more wire winds can separate the two elements, or the elements 154 can be spaced directly adjacent each either at adjacent winds.

FIG. 15 illustrates the winding pattern of a DFT stent in more detail, where the one or more wires comprising the DFT stent are wound in an over-under pattern. This pattern creates a plurality of diamond shaped cells along the length of the stent, shown in FIGS. 12 and 15. The black wires reflect several winds of a wire along a first direction (e.g., left to right along the mandrel) while the gray wires reflect several winds of a wire along a second direction (e.g., right to left along the mandrel).

As is shown, the winds of the wire are woven in an over-under pattern. In the context of FIG. 15, wire wind element 164a first is wound over element 162a, then under the next element 162b, then over the next element 162c, etc. Some sort of over-under pattern is necessary in order to prevent the stent from unraveling, however, various embodiments can use different over-under patterns (e.g. over one wire segment then under two wire segments or vice-versa, over one wire segment then under three wire segments or vice-versa, etc.).

Since there is some sort of over-under pattern used to wind the stent, as discussed above, if the reinforcing element 154 used is sufficiently long, the reinforcing element will also proceed over and under particular wire segments. For example, if a reinforcing element 154 is used along wire element 164b of FIG. 15 and extends over the entire length of the wire element 164b length shown, the reinforcing element will also proceed in an over-under pattern along with its associated wire 164b. The reinforcing element, due to its larger size, will serve to increase the spacing between the overlying or underlying wire segments, which helps augment wire movement as the stent expands in turn further helping the stent to adopt its expanded shape upon delivery from the catheter.

The earlier description referenced how the reinforcing element 154 can either be attached to the wire or float (meaning not connected to its associated wire region). Each design has its own merits. For instance, attaching the reinforcing element 154 can localize the desired expansion characteristics to particular segment(s) of the stent. However, not attaching the reinforcing element 154 (meaning it can “float” or has some degree of movement) can allow the reinforcing element some degree or “give” to make slight movement adjustments as needed, which can be beneficial during expansion when the stent is exposed to complex forces from various directions.

Note, although the reinforcing element 154 is shown along a defined segment along one “face” of the stent in FIG. 12, the reinforcing element 154 can be sized longer or shorter as needed. Therefore, a longer reinforcing element 154 can span multiple winds/picks/revolutions of the wire(s) comprising the DFT stent.

Although the reinforcing element 154 and its associated benefits have been discussed with regard to the DFT stent embodiments presented herein, the reinforcing element 154 can be also be incorporated along more traditional (non-DFT) stents and has its own utility in modulating stiffness along one or more sections of a traditional stent. In other words, this idea can also be used with other stent designs in order to incorporate these benefits into other stent designs.

The DFT stent, as described in the embodiments presented thus far, can be used for a variety of purposes (e.g., propping open a blood vessel, diverting flow from a target region such as an aneurysm, or retaining embolic within a target region). In one embodiment, the DFT stent is a coil-assist stent which serves as a scaffold to retain embolic coils within a target treatment region such as an aneurysm in order to retain the embolic material within the aneurysm treatment site. In one example, the pores of the DFT stent (the diamond shaped patterns of FIG. 15) are sized to allow a microcatheter to pass therethrough such that the DFT stent is first placed adjacent the aneurysm, then a microcatheter is delivered through a pore of the DFT stent into the aneurysm, where embolic material is then delivered through the microcatheter and into the aneurysm to occlude the aneurysm. In one example, the pores are sized from about 0.3-0.5 mm when the stent is in its expanded configuration.

The discussion above included references to the short flares 118 and how, in some embodiments, the short flares would be considered part of the “working length” of the stent. In these embodiments, the pore size of the short flares would be similar to the pore size of the rest of the stent.

A stent's PPI is generally calculated as the number of wire crossings or “picks” per inch along the length of a stent. The stent's PPI would depend on its intended purpose. For instance, a flow diversion stent would have a relatively large PPI since a denser wire cross-section is necessary to divert blood flow away from a target region (e.g., aneurysm). On the other hand, a coil-assist stent which serves to retain embolic material within a target region (e.g., aneurysm) would generally have a lower PPI since these stents serve more of a scaffolding function, and since larger pores may be needed to provide access for an embolic delivery catheter.

It should be understood that different aspects of the embodiments of this specification can be interchanged and combined with each other. In other words, additional embodiments are also specifically contemplated by combining different feature from different embodiments. Therefore, while specific embodiments are shown in the Figures, it is not intended that the invention necessarily be solely limited to those specific combinations.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A drawn-filled tubing (DFT) stent system comprising:
a DFT stent formed from at least one DFT wire wound into a generally tubular shape having loops at a proximal end and a distal end; the loops including a plurality of larger loops and a plurality of smaller loops;
a catheter to deliver the DFT stent;
a pusher to deploy the DFT stent from the catheter; the pusher including a pair of enlarged bands disposed on a distal region of the pusher;
terminal ends of the loops at the proximal end of the DFT stent each being located between said enlarged bands when said DFT is in an undeployed state.

2. The DFT stent system of claim 1, wherein each of the loops are angled at about 60 degrees from a longitudinal axis along the DFT stent when the DFT stent is in an expanded configuration.

3. The DFT stent system of claim 1, wherein the DFT stent is wound from solely one DFT wire.

4. The DFT stent system of claim 1, wherein the DFT wire comprises a platinum core and a nitinol jacket.

5. The DFT stent system of claim 1, wherein the DFT wire is about 0.0018-0.0022 inches in diameter.

6. The DFT stent system of claim 1, wherein the plurality of smaller loops are sized about 0.015-0.025 inches and the plurality of longer loops are sized about 0.04-0.045 inches.

7. The DFT stent system of claim 1, wherein the enlarged bands are spaced from each other by about 0.06-0.08 inches.

8. The DFT stent system of claim 1, wherein the loops at the proximal end of the DFT stent comprise three larger loops and three smaller loops, arranged in an alternating manner.

9. The DFT stent system of claim 8, wherein the larger loops include a marker coil.

10. A delivery system to deliver a drawn-filled tubing (DFT) stent:
a catheter;
a DFT stent carried inside said catheter, said DFT stent wound into a generally tubular shape and having proximal and distal loops; the proximal and distal loops including a plurality of larger loops and a plurality of smaller loops;
a pusher associated with said catheter; the pusher having a pair of bands disposed on a distal section of the pusher;
said bands being spaced from each other a distance, said distance accommodating a length of said plurality of larger and smaller loops located at a proximal end of said DFT stent when said DFT stent is carried in said catheter.

11. The delivery system of claim 10, wherein each of the loops of the DFT stent are angled at about 60 degrees from a longitudinal axis along the DFT stent when the DFT stent is in an expanded configuration.

12. The delivery system of claim 10, wherein the DFT stent is wound solely from one DFT wire.

13. The delivery system of claim 10, wherein the DFT wire of the DFT stent comprises a platinum core and a nitinol jacket.

14. The delivery system of claim 10, wherein the proximal loops of the DFT stent comprise four larger loops and four smaller loops, arranged in an alternating manner.

15. The delivery system of claim 14, wherein the larger loops include a marker coil.

16. A drawn-filled tubing (DFT) stent comprising:
at least one DFT wire wound into a generally tubular shape having loops at a proximal end of said tubular shape; the loops including a larger loop and a smaller loop;
said loops having a length when said DFT stent is in a compressed state that is sized for mating with a gap of a delivery pusher.

17. The DFT stent of claim 16, wherein each of the loops are angled at about 60 degrees from a longitudinal axis along the DFT stent when the DFT stent is in an expanded configuration.

18. The DFT stent of claim 16, wherein the DFT stent is wound solely from one DFT wire.

19. The DFT stent of claim 16, wherein the DFT wire comprises a platinum core and a nitinol jacket.

20. The DFT stent of claim 16, further comprising a coil wound around the DFT wire in a tubular section of the DFT stent.
